(19) United States
(12) Patent Application Publication
(10) Pub. No.: US 2021/0386745 A1
(43) Pub. Date: Dec. 16, 2021

(54) METHODS FOR TREATING SCHIZOPHRENIA

(71) Applicant: Indivior UK Limited, Hull (GB)

(72) Inventors: Christian A. HEIDBREDER, North Chesterfield, VA (US); Anne C. ANDORN, North Chesterfield, VA (US); James A. GRAHAM, North Chesterfield, VA (US)

(21) Appl. No.: 17/424,321

(22) PCT Filed: Jan. 21, 2020
(86) PCT No.: PCT/US2020/014461

§ 371 Date: Jul. 20, 2021

Related U.S. Application Data
(60) Provisional application No. 62/795,260, filed on Jan. 22, 2019.

(51) Int. Cl. A61K 31/519; A61K 47/32; A61K 47/34; A61K 9/00; A61P 25/18

(52) U.S. Cl. CPC A61K 31/519; A61K 47/32; A61P 25/18; A61K 9/0019; A61K 47/34

(57) ABSTRACT

The disclosure provides methods for treating negative, positive, and general symptoms of schizophrenia in patients using long-acting, injectable antipsychotic drugs. The disclosure provides methods of treating a negative symptom in a schizophrenic patient in need thereof by administering to the patient a therapeutically effective amount of a risperidone composition; wherein the risperidone composition comprises about 120 mg of risperidone base.

### CROSS-REFERENCE TO RELATED APPLICATIONS

This application claims priority to U.S. Application No. 62/795,260, filed Jan. 22, 2019, the disclosure of which is incorporated herein by reference in its entirety.

### BACKGROUND

Schizophrenia is a chronic serious mental illness that can be associated with significant disability. Medication is the only treatment for this disorder. There is a need for pharmaceutical compositions that deliver clinically relevant concentrations of antipsychotic drugs to the patients as quickly and simply as possible in order to provide antipsychotic efficacy during an acute exacerbation of psychosis. To improve treatment adherence and maintenance of clinically relevant antipsychotic concentrations, long-acting injectable antipsychotic medications have been approved by the United States Food and Drug Administration, including INVEGA SUSTENNA® (paliperidone palmitate, Janssen Pharmaceuticals, Inc.), INVEGA TRINZA® (paliperidone palmitate, Janssen Pharmaceuticals, Inc.), RISPERDAL CONSTA® (risperidone, Janssen Pharmaceuticals, Inc.), ARISTADA® (aripiprazole lauroxil, Alkermes, Inc.), ABILIFY MAINTENA® (aripiprazole, Otsuka Pharmaceutical Company), and ZYPREXA® RELPREVV® (olanzapine pamoate, Lilly, Inc.). In addition to long-acting injectable antipsychotic medications, there is also a supplemental injectable medication called ARISTADA INITIO® (aripiprazole lauroxil, Alkermes, Inc.) that is used to supplement treatment with ARISTADA®.

There is a need in the art for long-acting injectable antipsychotic medications that treat and relieve the debilitating symptoms of schizophrenia. The disclosure provides a solution to this need in the art.

### SUMMARY

The disclosure provides methods of treating a negative symptom in a schizophrenic patient in need thereof by administering to the patient a therapeutically effective amount of a risperidone composition; wherein the risperidone composition comprises about 120 mg of risperidone base.

The disclosure provides methods of treating a positive symptom in a schizophrenic patient in need thereof by administering to the patient a therapeutically effective amount of a risperidone composition; wherein the risperidone composition comprises about 120 mg of risperidone base; and wherein the positive symptom is hallucinatory behavior, conceptual disorganization, delusions, suspiciousness/persecution, hostility, excitement, or a combination of two or more thereof.

The disclosure provides methods of treating a general symptom in a schizophrenic patient in need thereof by administering to the patient a therapeutically effective amount of a risperidone composition; wherein the risperidone composition comprises about 120 mg of risperidone base; and wherein the general symptom is poor impulse control, somatic concern, depression, active social avoidance, uncooperativeness, poor attention, anxiety, tension, or a combination of two or more thereof.

The disclosure provides methods of treating a positive symptom in a schizophrenic patient in need thereof by administering to the patient a therapeutically effective amount of a risperidone composition; wherein the risperidone composition comprises about 90 mg of risperidone base; and wherein the positive symptom is hallucinatory behavior, conceptual disorganization, delusions, suspiciousness/persecution, or a combination of two or more thereof.

The disclosure provides methods of treating a general symptom in a schizophrenic patient in need thereof by administering to the patient a therapeutically effective amount of a risperidone composition; wherein the risperidone composition comprises about 90 mg of risperidone base; and wherein the general symptom is motor retardation, depression, active social avoidance, uncooperativeness, poor attention, anxiety, tension, or a combination of two or more thereof.

These and other embodiments are described herein.

### BRIEF DESCRIPTION OF THE DRAWINGS

FIG. 1 shows Forest Plots of PANSS Positive Item Analysis at day 57 (change from baseline) ITT population. The ITT population includes all randomized subjects who received at least one dose of RBP-7000 or placebo during the double-blind treatment period and had at least one post-baseline total PANSS score such that change from baseline could be calculated.

FIG. 2 shows Forest Plots of PANSS Negative Item Analysis at day 57 (change from baseline) ITT population. The ITT population includes all randomized subjects who received at least one dose of RBP-7000 or placebo during the double-blind treatment period and had at least one post-baseline total PANSS score such that change from baseline could be calculated.

### DETAILED DESCRIPTION

### Definitions

“Psychiatric disease” refers to any disease in the Diagnostic and Statistical Manual of Mental Disorders, Fifth Edition (DSM-5), the disclosure of which is incorporated by reference herein. In embodiments, the psychiatric disease is schizophrenia. In embodiments, the psychiatric disease is bipolar disorder. In embodiments, the psychiatric disease is bipolar mania. In embodiments, the psychiatric disease is autism. In embodiments, the psychiatric disease is anxiety disorder, social phobia, attention-deficit hyperactivity disorder, depression, an eating disorder, insomnia, obsessive-compulsive disorder, personality disorder, post-traumatic stress disorder, substance abuse, or Tourette's syndrome.

“Schizophrenia” is a psychiatric disease characterized by, e.g., delusions, hallucinations, disorganized speech, grossly disorganized or catatonic behavior, and negative symptoms. Schizophrenia can be acute schizophrenia. Diagnostic criteria for schizophrenia are set forth in the Diagnostic and Statistical Manual of Mental Disorders, 5th Edition, by the American Psychiatric Association, the disclosures of which are incorporated by reference herein.

“Schizophrenic patient” refers to a patient with schizophrenia.

“Positive and Negative Syndrome Scale” or “PANSS” refers to the medical assessment scale used to measure the symptoms and severity of schizophrenic patients. The scale consists of 30 items, where each item is scored from 1 through 7, where a score of 1 indicates the absence of the symptom, and a score of 7 indicates extreme suffering from the symptom. These 30 items are grouped into 3 subscales: positive symptoms (7 items), negative symptoms (7 items), and general psychopathology (16 items). PANSS is further described in Kay et al, “The positive and negative syndrome scale (PANS S) for schizophrenia,” Schizophr. Bull., 13(2):261-276 (1987). In the methods of treatment described herein, the total PANSS score from baseline is decreased by at least 10%. In the methods of treatment described herein, the total PANSS score from baseline is decreased by at least 15%. In the methods of treatment described herein, the total PANSS score from baseline is decreased by at least 20%. In the methods of treatment described herein, the total PANSS score from baseline is decreased by at least 25%. In the methods of treatment described herein, the total PANSS score from baseline is decreased by at least 30%. In the methods of treatment described herein, the total PANSS score from baseline is decreased by at least 35%. In the methods of treatment described herein, the total PANSS score from baseline is decreased by at least 40%. In the methods of treatment described herein, the total PANSS score from baseline is decreased by at least 45%. In the methods of treatment described herein, the total PANSS score from baseline is decreased by at least 50%.

“Positive symptoms” of schizophrenia refer to the positive symptoms on the Positive and Negative Syndrome Scale. The positive symptoms include delusions, conceptual disorganization, hallucinations, excitement, grandiosity, suspiciousness/persecution, and hostility. The minimum PANSS score for positive symptoms is 7, and the maximum score is 49.

“Negative symptoms” of schizophrenia refer to the negative symptoms on the Positive and Negative Syndrome Scale. The negative symptoms include blunted affect, emotional withdrawal, poor rapport, passive/apathetic social withdrawal, difficulty in abstract thinking, lack of spontaneity and flow of conversation, and stereotyped thinking. The minimum PANSS score for negative symptoms is 7, and the maximum score is 49.

“General symptoms” of schizophrenia refer to the general psychopathology symptoms on the Positive and Negative Syndrome Scale. The general symptoms include somatic concern, anxiety, guilt feelings, tension, mannerisms and posturing, depression, motor retardation, uncooperativeness, unusual though content, disorientation, poor attention, lack of judgment and insight, disturbance of volition, poor impulse control, preoccupation, and active social avoidance. The minimum PANSS score for general symptoms is 16, and the maximum score is 112.

“Long-acting injectable antipsychotic medication” refers to any long-acting injectable antipsychotic medication. “Long-acting” refers to a medication that can be administered once per week; once every two weeks; once every three weeks; once per month; once every six weeks; once every other month; once every three months, and the like.

“Injectable” refers to any form of parenteral injection, such as subcutaneous injection, intramuscular injection, intravenous injection, and the like. Exemplary long-acting injectable antipsychotic medications include the risperidone compositions described herein, INVEGA SUSTENNA®, INVEGA TRINZA®, RISPERDAL CONSTA®, ARISTADA®, ABILIFY MAINTENA®, and ZYPREXA® RELPREVV®.

“Risperidone compositions described herein” and “risperidone composition” refer to the long-acting injectable risperidone compositions of Formulation A, Formulation B, Formulation C, Formulation D, and variations thereof, as described herein.

“Formulation A” refers to a risperidone composition which comprises about 5 wt % to about 25 wt % risperidone base; about 25 wt % to about 50 wt % of a poly(lactide-co-glycolide) copolymer; and about 35 wt % to about 60 wt % N-methyl-2-pyrrolidone. In aspects, the poly(lactide-co-glycolide) copolymer is a 50:50 to 90:10 poly(lactide-co-glycolide) copolymer. In aspects, the poly(lactide-co-glycolide) copolymer is a 80:20 poly(lactide-co-glycolide) copolymer. In aspects, the poly(lactide-co-glycolide) copolymer is a 75:25 poly(lactide-co-glycolide) copolymer. In aspects, the poly(lactide-co-glycolide) copolymer is a 85:15 poly(lactide-co-glycolide) copolymer. In aspects, the poly(lactide-co-glycolide) copolymer has a number average molecular weight from about 1,000 Daltons to about 50,000 Daltons. In aspects, the poly(lactide-co-glycolide) copolymer has a number average molecular weight from about 10,000 Daltons to about 40,000 Daltons. In aspects, the poly(lactide-co-glycolide) copolymer has a number average molecular weight from about 20,000 Daltons to about 30,000 Daltons. In aspects, the poly(lactide-co-glycolide) copolymer has a number average molecular weight from about 23,000 Daltons to about 26,000 Daltons.

In aspects, the poly(lactide-co-glycolide) copolymer comprises a carboxy terminal group. Formulation A can be prepared by processes known in the art and described, for example, in U.S. Pat. Nos. 9,180,197, 9,186,413, and 10,010,612, the disclosures of which are incorporated by reference herein in their entirety.

“Formulation B” refers to a risperidone composition which comprises about 10 wt % to about 20 wt % risperidone base; about 35 wt % to about 45 wt % of a poly(lactide-co-glycolide) copolymer; and about 40 wt % to about 50 wt % N-methyl-2-pyrrolidone. In aspects, the poly(lactide-co-glycolide) copolymer is a 50:50 to 90:10 poly(lactide-co-glycolide) copolymer. In aspects, the poly(lactide-co-glycolide) copolymer is a 80:20 poly(lactide-co-glycolide) copolymer. In aspects, the poly(lactide-co-glycolide) copolymer is a 75:25 poly(lactide-co-glycolide) copolymer. In aspects, the poly(lactide-co-glycolide) copolymer is a 85:15 poly(lactide-co-glycolide) copolymer. In aspects, the poly(lactide-co-glycolide) copolymer has a number average molecular weight from about 1,000 Daltons to about 50,000 Daltons. In aspects, the poly(lactide-co-glycolide) copolymer has a number average molecular weight from about 10,000 Daltons to about 40,000 Daltons. In aspects, the poly(lactide-co-glycolide) copolymer has a number average molecular weight from about 20,000 Daltons to about 30,000 Daltons. In aspects, the poly(lactide-co-glycolide) copolymer has a number average molecular weight from about 23,000 Daltons to about 26,000 Daltons.

In aspects, the poly(lactide-co-glycolide) copolymer comprises a carboxy terminal group. Formulation B can be prepared by processes known in the art and described, for example, in U.S. Pat. Nos. 9,180,197, 9,186,413, and 10,010,612, the disclosures of which are incorporated by reference herein in their entirety.

“Formulation C” refers to a risperidone composition which comprises about 15 wt % risperidone base; about 38 wt % of an 80:20 poly(lactide-co-glycolide) copolymer; and about 47 wt % N-methyl-2-pyrrolidone. In aspects, the poly(lactide-co-glycolide) copolymer has a number average molecular weight from about 20,000 Daltons to about 30,000 Daltons. In aspects, the poly(lactide-co-glycolide) copolymer comprises a carboxy terminal group. Formulation C can be prepared by processes known in the art and described, for example, in U.S. Pat. Nos. 9,180,197, 9,186,413, and 10,010,612, the disclosures of which are incorporated by reference herein in their entirety.

“Formulation D” refers to a risperidone composition which comprises about 1 wt % to about 30 wt % of risperidone or a pharmaceutically acceptable salt of risperidone; about 10 wt % to about 80 wt % of a biodegradable polymer; and about 10 wt % to about 80 wt % of an organic solvent. In embodiments, the biodegradable polymer is a polylactide, a polyglycolide, a polycaprolactone, a copolymer thereof, a terpolymer thereof, or any combination thereof. In aspects, the biodegradable polymer is a polylactide polymer. In aspects, the biodegradable polymer is a polyglycolide polymer. In aspects, the biodegradable polymer is a poly(lactide-co-glycolide) copolymer. In aspects, the poly(lactide-co-glycolide) copolymer is a 10:90 to 95:5 poly(lactide-co-glycolide) copolymer. In aspects, the poly(lactide-co-glycolide) copolymer is a 50:50 to 90:10 poly(lactide-co-glycolide) copolymer. In aspects, the poly(lactide-co-glycolide) copolymer is a 80:20 poly(lactide-co-glycolide) copolymer. In aspects, the poly(lactide-co-glycolide) copolymer is a 75:25 poly(lactide-co-glycolide) copolymer. In aspects, the poly(lactide-co-glycolide) copolymer is a 85:15 poly(lactide-co-glycolide) copolymer. In aspects, the poly(lactide-co-glycolide) copolymer has a number average molecular weight from about 1,000 Daltons to about 50,000 Daltons. In aspects, the poly(lactide-co-glycolide) copolymer has a number average molecular weight from about 10,000 Daltons to about 40,000 Daltons. In aspects, the poly(lactide-co-glycolide) copolymer has a number average molecular weight from about 20,000 Daltons to about 30,000 Daltons. In aspects, the poly(lactide-co-glycolide) copolymer has a number average molecular weight from about 23,000 Daltons to about 26,000 Daltons. In aspects, the poly(lactide-co-glycolide) copolymer comprises a carboxy terminal group. In embodiments, the organic solvent is N-methyl-2-pyrrolidone, 2-pyrrolidone, acetic acid, lactic acid, methyl lactate, ethyl lactate, monomethyl succinate acid, monomethyl citric acid, glycofurol, glycerol formal, isopropylidene glycol, 2,2-dimethyl-1,3-dioxolone-4-methanol, solketal, dimethylformamide, dimethylacetamide, dimethylsulfoxide, dimethyl sulfone, epsilon-caprolactone, butyrolactone, caprolactam, or a mixture of two or more thereof. In embodiments, the organic solvent is N-methyl-2-pyrrolidone. Formulation D can be prepared by processes known in the art and described, for example, in U.S. Pat. Nos. 9,180,197, 9,186,413, and 10,010,612, the disclosures of which are incorporated by reference herein in their entirety.

“Number average molecular weight” refers to the total weight of all the polymer molecules in a sample, divided by the total number of polymer molecules in a sample. Number average molecular weight can be determined by methods known in the art, such as by gel permeation chromatography/size exclusion chromatography (e.g., available from Agilent Technologies).

“Therapeutically effective amount” refers to an amount of the drug sufficient to contribute to the treatment or reduction of a symptom or symptoms of a psychiatric disease, such as schizophrenia.

“Administering” refers to intravenous, parenteral, intraperitoneal, intramuscular, intrathecal, intracranial, or subcutaneous administration, or the implantation of a slow-release device (e.g., a solid polymeric biodegradable device, a mini-osmotic pump) to a subject.

Parenteral administration includes, e.g., intravenous, intramuscular, intra-arteriole, intradermal, subcutaneous, intraperitoneal, intraventricular, and intracranial. In embodiments, parenteral administration is subcutaneous administration. Other modes of delivery include, but are not limited to, the use of microsphere formulations, liposomal formulations, intravenous infusion, etc. The compositions may additionally include components to provide sustained release and/or comfort. Such components include high molecular weight, anionic mucomimetic polymers, gelling polysaccharides and finely-divided drug carrier substrates. The compositions can also be delivered as microspheres for slow release in the body. For example, microspheres can be administered via intradermal injection of drug-containing microspheres, which slowly release intramuscularly or subcutaneously; as biodegradable and injectable gel formulations; or as microspheres for oral administration. The compositions can also be delivered as nanoparticles.

“Treating” or “treatment” refers to any indicia of success in the treatment or amelioration of a psychiatric disease, including any objective or subjective parameter such as abatement; remission; diminishing of symptoms or making the condition more tolerable to the human; slowing in the rate of degeneration or decline; making the final point of degeneration less debilitating; improving a human's physical or mental well-being. The success in the treatment or amelioration of symptoms can be based on objective or subjective parameters; including the results of a physical examination, neuropsychiatric exams, and/or a psychiatric evaluation.

“Month” means 28 days to 31 days. In one embodiment, a month is 28 days, 29 days, 30 days, or 31 days. In one embodiment, a month is 28 days. In one embodiment, a month is 30 days. In one embodiment, a month is 31 days.

Methods

The disclosure provides methods of treating a negative symptom in a schizophrenic patient in need thereof, the method comprising administering to the patient a therapeutically effective amount of a risperidone composition; wherein the risperidone composition comprises about 120 mg of risperidone base. In aspects, the negative symptom is emotional withdrawal. In aspects, the negative symptom is passive/apathetic social withdrawal. In aspects, the negative symptom is difficulty in abstract thinking. In aspects, the negative symptom is stereotyped thinking. In aspects, the negative symptoms are emotional withdrawal and passive/apathetic social withdrawal. In aspects, the negative symptoms are emotional withdrawal, passive/apathetic social withdrawal, and difficulty in abstract thinking.

In aspects, the negative symptom is emotional withdrawal, passive/apathetic social withdrawal, and stereotyped thinking, or a combination of two or more thereof. In aspects, the negative symptom is emotional withdrawal, passive/apathetic social withdrawal, difficulty in abstract thinking, and stereotyped thinking. In aspects, the negative symptom is emotional withdrawal, passive/apathetic social withdrawal, difficulty in abstract thinking, stereotyped thinking, or a combination of two or more thereof. In aspects, the negative symptom is blunted affect, emotional withdrawal, poor rapport, passive/apathetic social withdrawal, difficulty in abstract thinking, lack of spontaneity and flow of conversation, stereotyped thinking, or a combination of two or more thereof. In aspects, the negative symptom is blunted affect. In aspects, the negative symptom is poor rapport. In aspects, the negative symptom is lack of spontaneity and flow of conversation. In aspects, the methods of treating negative symptoms in a schizophrenic patient are methods of reducing the PANSS score for the negative symptoms in the schizophrenic patient. The reduction is PANSS score is based on the change from the baseline assessment of the patient. In aspects, the PANSS is reduced by at least 14 relative to baseline. In aspects, the PANSS is reduced by at least 15. In aspects, the PANSS is reduced by at least 16. In aspects, the PANSS is reduced by at least 17. In aspects, the PANSS is reduced by at least 18. In aspects, the PANSS is reduced by at least 19. In aspects, the PANSS is reduced by at least 20. In aspects, the PANSS is reduced by at least 21. In aspects, the PANSS is reduced by at least 22. In aspects, the PANSS is reduced by at least 23. In aspects, the PANSS is reduced by at least 24. In aspects, the PANSS is reduced by at least 25. In aspects, the PANSS is reduced by at least 26. In aspects, the PANSS is reduced by at least 27. In aspects, the PANSS is reduced by at least 28. In aspects, the PANSS is reduced by at least 29. In aspects, the PANSS is reduced by at least 30. In aspects, the PANSS is reduced by more than 30. The reduction in PANSS score is based on the change from the baseline assessment of the patient.

The disclosure provides methods of treating a positive symptom in a schizophrenic patient in need thereof, the method comprising administering to the patient a therapeutically effective amount of a risperidone composition; wherein the risperidone composition comprises about 120 mg of risperidone base; and wherein the positive symptom is hallucinatory behavior, conceptual disorganization, delusions, suspiciousness/persecution, hostility, excitement, or a combination of two or more thereof. In aspects, the positive symptom is hallucinatory behavior. In aspects, the positive symptom is conceptual disorganization. In aspects, the positive symptom is delusions. In aspects, the positive symptom is suspiciousness/persecution. In aspects, the positive symptom is hostility. In aspects, the positive symptom is excitement. In aspects, the methods of treating positive symptoms in a schizophrenic patient are methods of reducing the PANSS score for the positive symptoms in the schizophrenic patient. The reduction is PANSS score is based on the change from the baseline assessment of the patient. In aspects, the PANSS is reduced by at least 14 relative to baseline. In aspects, the PANSS is reduced by at least 15. In aspects, the PANSS is reduced by at least 16. In aspects, the PANSS is reduced by at least 17. In aspects, the PANSS is reduced by at least 18. In aspects, the PANSS is reduced by at least 19. In aspects, the PANSS is reduced by at least 20. In aspects, the PANSS is reduced by at least 21. In aspects, the PANSS is reduced by at least 22. In aspects, the PANSS is reduced by at least 23. In aspects, the PANSS is reduced by at least 24. In aspects, the PANSS is reduced by at least 25. In aspects, the PANSS is reduced by at least 26. In aspects, the PANSS is reduced by at least 27. In aspects, the PANSS is reduced by at least 28. In aspects, the PANSS is reduced by at least 29. In aspects, the PANSS is reduced by at least 30. In aspects, the PANSS is reduced by more than 30. The reduction in PANSS score is based on the change from the baseline assessment of the patient.

The disclosure provides methods of treating a general symptom in a schizophrenic patient in need thereof, the method comprising administering to the patient a therapeutically effective amount of a risperidone composition; wherein the risperidone composition comprises about 120 mg of risperidone base; and wherein the general symptom is poor impulse control, somatic concern, depression, active social avoidance, uncooperativeness, poor attention, anxiety, tension, or a combination of two or more thereof. In aspects, the general symptom is poor impulse control. In aspects, the general symptom is somatic concern. In aspects, the general symptom is depression. In aspects, the general symptom is active social avoidance. In aspects, the general symptom is uncooperativeness. In aspects, the general symptom is poor attention. In aspects, the general symptom is anxiety. In aspects, the general symptom is tension. In aspects, the methods of treating general symptoms in a schizophrenic patient are methods of reducing the PANSS score for the general symptoms in the schizophrenic patient. The reduction is PANSS score is based on the change from the baseline assessment of the patient. In aspects, the PANSS is reduced by at least 14 relative to baseline. In aspects, the PANSS is reduced by at least 15. In aspects, the PANSS is reduced by at least 16. In aspects, the PANSS is reduced by at least 17. In aspects, the PANSS is reduced by at least 18. In aspects, the PANSS is reduced by at least 19. In aspects, the PANSS is reduced by at least 20. In aspects, the PANSS is reduced by at least 21. In aspects, the PANSS is reduced by at least 22. In aspects, the PANSS is reduced by at least 23. In aspects, the PANSS is reduced by at least 24. In aspects, the PANSS is reduced by at least 25. In aspects, the PANSS is reduced by at least 26. In aspects, the PANSS is reduced by at least 27. In aspects, the PANSS is reduced by at least 28. In aspects, the PANSS is reduced by at least 29. In aspects, the PANSS is reduced by at least 30. In aspects, the PANSS is reduced by more than 30. The reduction in PANSS score is based on the change from the baseline assessment of the patient.

In aspects of the methods of treating negative symptoms, positive symptoms, and general symptoms in a schizophrenic patient in need thereof, the patient is administered a risperidone composition which comprises about 120 mg of risperidone base; a poly(lactide-co-glycolide) copolymer; and N-methyl-2-pyrrolidone. In aspects, the method is for treating negative symptoms. In aspects, the method is for treating positive symptoms. In aspects, the method is for treating general symptoms. In aspects, the risperidone composition is Formulation A. In aspects, the risperidone composition is Formulation B. In aspects, the risperidone composition is Formulation C. In aspects, the risperidone composition is Formulation D. In aspects, the risperidone composition comprises risperidone base at a concentration of about 5 wt % to about 25 wt %; about 25 wt % to about 50 wt % of a poly(lactide-co-glycolide) copolymer; and about 35 wt % to about 60 wt % of N-methyl-2-pyrrolidone. In aspects, the poly(lactide-co-glycolide) copolymer is a 50:50 to 90:10 poly(lactide-co-glycolide)copolymer. In aspects, the poly(lactide-co-glycolide) copolymer is an 80:20 poly(lactide-co-glycolide) copolymer. In aspects, the poly(lactide-co-glycolide) copolymer has a weight average molecular weight from about 20,000 Daltons to about 30,000 Daltons. In aspects, the risperidone composition comprises risperidone base at a concentration of about 10 wt % to about 20 wt %; about 35 wt % to about 45 wt % of a 50:50 to 90:10 poly(lactide-co-glycolide) copolymer; and about 40 wt % to about 50 wt % of N-methyl-2-pyrrolidone. In aspects, the risperidone composition comprises about 15 wt % risperidone base; about 38 wt % of a poly(lactide-co-glycolide)copolymer; and about 47 wt % N-methyl-2-pyrrolidone. In aspects, the risperidone composition comprises about 15 wt % risperidone base; about 38 wt % of an 80:20 poly(lactide-co-glycolide)copolymer; and about 47 wt % N-methyl-2-pyrrolidone. In aspects, the risperidone composition is administered to the patient by subcutaneous injection. In aspects, the risperidone composition is administered to the patient by subcutaneous injection once per month. In aspects, the risperidone composition is administered to the patient by subcutaneous injection once every two months. In aspects, the risperidone composition is administered to the patient by subcutaneous injection once every three months.

The disclosure provides methods of treating a positive symptom in a schizophrenic patient in need thereof, the method comprising administering to the patient a therapeutically effective amount of a risperidone composition; wherein the risperidone composition comprises about 90 mg of risperidone base; and wherein the positive symptom is hallucinatory behavior, conceptual disorganization, delusions, suspiciousness/persecution, or a combination of two or more thereof. In aspects, the positive symptom is hallucinatory behavior. In aspects, the positive symptom is conceptual disorganization. In aspects, the positive symptom is delusions. In aspects, the positive symptom is suspiciousness/persecution. In aspects, the methods of treating positive symptoms in a schizophrenic patient are methods of reducing the PANSS score for the positive symptoms in the schizophrenic patient. The reduction is PANSS score is based on the change from the baseline assessment of the patient. In aspects, the PANSS is reduced by at least 14 relative to baseline. In aspects, the PANSS is reduced by at least 15. In aspects, the PANSS is reduced by at least 16. In aspects, the PANSS is reduced by at least 17. In aspects, the PANSS is reduced by at least 18. In aspects, the PANSS is reduced by at least 19. In aspects, the PANSS is reduced by at least 20. In aspects, the PANSS is reduced by at least 21. In aspects, the PANSS is reduced by at least 22. In aspects, the PANSS is reduced by at least 23. In aspects, the PANSS is reduced by at least 24. In aspects, the PANSS is reduced by at least 25. In aspects, the PANSS is reduced by at least 26. In aspects, the PANSS is reduced by at least 27. In aspects, the PANSS is reduced by at least 28. In aspects, the PANSS is reduced by at least 29. In aspects, the PANSS is reduced by at least 30. In aspects, the PANSS is reduced by more than 30. The reduction in PANSS score is based on the change from the baseline assessment of the patient.

The disclosure provides methods of treating a general symptom in a schizophrenic patient in need thereof, the method comprising administering to the patient a therapeutically effective amount of a risperidone composition; wherein the risperidone composition comprises about 90 mg of risperidone base; and wherein the general symptom is motor retardation, depression, active social avoidance, uncooperativeness, poor attention, anxiety, tension, or a combination of two or more thereof. In aspects, the general symptom is motor retardation. In aspects, the general symptom is depression. In aspects, the general symptom is active social avoidance. In aspects, the general symptom is uncooperativeness. In aspects, the general symptom is poor attention. In aspects, the general symptom is anxiety. In aspects, the general symptom is tension. In aspects, the methods of treating general symptoms in a schizophrenic patient are methods of reducing the PANSS score for the general symptoms in the schizophrenic patient. The reduction is PANSS score is based on the change from the baseline assessment of the patient. In aspects, the PANSS is reduced by at least 14 relative to baseline. In aspects, the PANSS is reduced by at least 15. In aspects, the PANSS is reduced by at least 16. In aspects, the PANSS is reduced by at least 17. In aspects, the PANSS is reduced by at least 18. In aspects, the PANSS is reduced by at least 19. In aspects, the PANSS is reduced by at least 20. In aspects, the PANSS is reduced by at least 21. In aspects, the PANSS is reduced by at least 22. In aspects, the PANSS is reduced by at least 23. In aspects, the PANSS is reduced by at least 24. In aspects, the PANSS is reduced by at least 25.

In aspects, the PANSS is reduced by at least 26. In aspects, the PANSS is reduced by at least 27. In aspects, the PANSS is reduced by at least 28. In aspects, the PANSS is reduced by at least 29. In aspects, the PANSS is reduced by at least 30. In aspects, the PANSS is reduced by more than 30. The reduction in PANSS score is based on the change from the baseline assessment of the patient.

In aspects of the methods of treating positive symptoms and general symptoms in a schizophrenic patient in need thereof, the patient is administered a risperidone composition which comprises about 90 mg of risperidone base; a poly(lactide-co-glycolide) copolymer; and N-methyl-2-pyrrolidone. In aspects, the method is for treating positive symptoms. In aspects, the method is for treating general symptoms. In aspects, the risperidone composition is Formulation A. In aspects, the risperidone composition is Formulation B. In aspects, the risperidone composition is Formulation C. In aspects, the risperidone composition is Formulation D. In aspects, the risperidone composition comprises risperidone base at a concentration of about 5 wt % to about 25 wt %; about 25 wt % to about 50 wt % of a poly(lactide-co-glycolide) copolymer; and about 35 wt % to about 60 wt % of N-methyl-2-pyrrolidone. In aspects, the poly(lactide-co-glycolide) copolymer is a 50:50 to 90:10 poly(lactide-co-glycolide)copolymer. In aspects, the poly(lactide-co-glycolide) copolymer is an 80:20 poly(lactide-co-glycolide) copolymer. In aspects, the poly(lactide-co-glycolide) copolymer has a weight average molecular weight from about 20,000 Daltons to about 30,000 Daltons. In aspects, the risperidone composition comprises risperidone base at a concentration of about 10 wt % to about 20 wt %; about 35 wt % to about 45 wt % of a 50:50 to 90:10 poly(lactide-co-glycolide) copolymer; and about 40 wt % to about 50 wt % of N-methyl-2-pyrrolidone. In aspects, the risperidone composition comprises about 15 wt % risperidone base; about 38 wt % of a poly(lactide-co-glycolide)copolymer; and about 47 wt % N-methyl-2-pyrrolidone. In aspects, the risperidone composition comprises about 15 wt % risperidone base; about 38 wt % of an 80:20 poly(lactide-co-glycolide)copolymer; and about 47 wt % N-methyl-2-pyrrolidone. In aspects, the risperidone composition is administered to the patient by subcutaneous injection. In aspects, the risperidone composition is administered to the patient by subcutaneous injection once per month. In aspects, the risperidone composition is administered to the patient by subcutaneous injection once every two months. In aspects, the risperidone composition is administered to the patient by subcutaneous injection once every three months.

### EXAMPLES

The following examples are for purposes of illustration only and are not intended to limit the spirit or scope of the disclosure or claims.

### Example 1

A phase 3, randomized, double-blind, placebo-controlled, multicenter clinical study (NCT02109562) was conducted to evaluate the efficacy, safety and tolerability of Formulation C (90 mg and 120 mg) as a treatment in subjects with acute schizophrenia, subjects in an acute psychotic state, or subjects in relapse with acute schizophrenic symptoms, who had a PANSS score of at least 80-120 and a score greater than 4 on at least two of the following four items: hallucinatory behavior, delusions, conceptual disorganization, or suspiciousness/persecution at screening. Subjects were randomly assigned to receive 2 subcutaneous doses of either Formulation C (90 or 120 mg) or placebo over 8 weeks. A total of 538 subjects were screened to enroll in this Phase 3 study. Only 354 subjects passed screening, of which 119 belonged to the placebo group and 116 and 119 belonged to the 90 mg and 120 mg treatment arms, respectively. Furthermore, 17 subjects (7-placebo, 5-90 mg, 5-120 mg) failed to make it to the Intent-to-Treat (ITT) group as they either did not receive at least one dose of Formulation C or did not have at least one assessment post-baseline that facilitates the calculation of change from baseline.

During the screening phase all subjects received 0.25 mg of oral risperidone at Visit 1 (3 to 8 days before double-blind treatment) and a second dose of 0.25 mg oral risperidone 24 hours later to assess their tolerability to risperidone prior to receiving Formulation C. Subjects who passed screening were tapered off their oral anti-psychotic if applicable. Tapering rates for washout medications were at the discretion of the investigator and were determined on an individual basis, with consideration to patient state, dose, and known PK of the medication being tapered, provided the restricted medication was discontinued during Day −8 and Day −1, inclusive. Modifications to subject's pre-existing treatment were not made unless deemed clinically important by the investigator. Upon completion of all study participation requirements, subjects were randomized to one of the three study treatments at Visit 3 (Day 1), corresponding to the start of the 8-week double-blind treatment period: subjects received two subcutaneous injections of Formulation C (90 mg or 120 mg) or placebo at a 28-day interval, on Day 1 and on Day 29.

Characteristics of the patient population, shown in Table 1, were balanced across the treatment groups. The mean baseline PANSS total score ranged from 94 to 96 across the groups. Most patients were male (74 to 83% per group), and the mean ages were 40 to 43 in each group. Most patients in this study were black or African American (71 to 75% per group). Of the 354 subjects randomized to treatment, 337 were included in the intent-to-treat (ITT) population, and 259 (73%) completed the study. Subgroup analyses by gender, age, and race did not suggest any clear evidence of differential responsiveness to Formulation C.

TABLE 1

| | | | 90 mg | | 120 mg | |
|---|---|---|---|---|---|---|
| | Placebo | | Formulation C | | Formulation C | |
| | n = 112 | | n = 111 | | n = 114 | |
| Characteristic | n | % | n | % | n | % |
| Gender | | | | | | |
| Male | 81 | 72.3 | 93 | 83.8 | 84 | 73.7 |
| Female | 31 | 27.7 | 18 | 16.2 | 30 | 26.3 |
| Race | | | | | | |
| White | 25 | 22.3 | 28 | 25.2 | 30 | 26.3 |
| Black | 84 | 75.0 | 79 | 71.2 | 80 | 70.2 |
| Asian | 1 | 0.9 | 1 | 0.9 | 3 | 2.6 |
| Native | 1 | 0.9 | 1 | 0.9 | 1 | 0.9 |
| Hawaiian or | | | | | | |
| Pacific Islander | | | | | | |
| Other | 1 | 0.9 | 2 | 1.8 | 0 | 0 |
| Ethnicity | | | | | | |
| Hispanic/Latino | 10 | 8.9 | 7 | 6.3 | 9 | 7.9 |
| Not Hispanic or | 101 | 90.2 | 104 | 93.7 | 104 | 91.2 |
| Latino | | | | | | |
| Unknown | 1 | 0.9 | 0 | 0 | 1 | 0.9 |
| | Mean | SD | Mean | SD | Mean | SD |
| Age at First | 26.6 | 9.25 | 25.5 | 8.21 | 26.9 | 8.48 |
| Schizophrenia | | | | | | |
| Diagnosis | | | | | | |
| (years) | | | | | | |
| PANSS | | | | | | |
| Total Score | 94.1 | 8.89 | 95.5 | 9.23 | 94.9 | 8.09 |
| Positive | 25.4 | 3.31 | 26.0 | 3.36 | 25.9 | 3.42 |
| symptom | | | | | | |
| subscale | | | | | | |
| Negative | 22.6 | 3.79 | 23.5 | 3.68 | 22.6 | 3.96 |
| symptom | | | | | | |
| subscale | | | | | | |
| General | 46.2 | 5.49 | 45.9 | 5.94 | 46.5 | 5.15 |
| psychopathology | | | | | | |
| subscale | | | | | | |
| CGI-S | 4.8 | 0.59 | 4.8 | 0.58 | 4.8 | 0.48 |

The clinical assessments for efficacy included the PANSS and the CGI-S scales. The PANSS scale consists of 30 items, where each item is scored from 1 through 7, where 1 indicates the absence of the symptom, and 7 indicates extreme suffering from the symptom. These 30 items are grouped into 3 subscales; positive (7 items), negative (7 items) and general psychopathology (16 items). PANSS scores were collected at baseline on Day 1 and on Days 15+/−1, 29, 43+/−1 and 57. A total of 1571 PANSS assessments were available for analysis from this 8-week study.

The primary endpoint was the change in PANSS total score from baseline to end of study (Day 57). Both Formulation C 90 and 120 mg doses demonstrated a statistically significant improvement compared with placebo based on the primary endpoint, as shown in Table 2.

TABLE 2

| | Primary Efficacy Measures: PANSS total score | | |
|---|---|---|---|
| | Mean | | Placebo- |
| | Baseline | LS Mean | Subtracted |
| Treatment Group | Score | Change from | Difference^{a} |
| N (#ITT subjects) | (SD) | Baseline (SE) | (95% CI) |
| Formulation C (90 mg)* | 95.5 (9.23) | −19.86 (1.56) | −6.50 |
| (N = 111) | | | (−10.87, −2.13)* |
| Formulation C (120 mg)* | 94.8 (8.09) | −23.61 (1.58) | −10.24 |
| (N = 114) | | | (−14.64, −5.85)* |
| Placebo | 94.1 (8.89) | −13.37(1.58) | — |
| (N = 112) | | | |

With reference to Table 2: “ITT” is intent-to-treat; “SD” is standard deviation; “SE” is standard error; “LS Mean” is least-square mean; “CI” is unadjusted confidence interval; “Difference^{a}” is the difference (drug minus placebo) in least-squares mean change from baseline; and * is a dose that is statistically significantly superior to placebo.

### Example 2

In the clinical study described in Example 1, a mixed-effects model for repeated measures was used to examine the primary endpoint. Compared with placebo, PANSS total score was significantly improved at Day 57 by treatment with either 90 mg of Formulation C (P<0.01) or 120 mg of Formulation C (P<0.0001). A post hoc analysis of the results of the clinical study described in Example 1 was conducted. A mixed-effects model for repeated measures was used to examine changes in the scores of the 30 PANSS items from baseline to week 8.

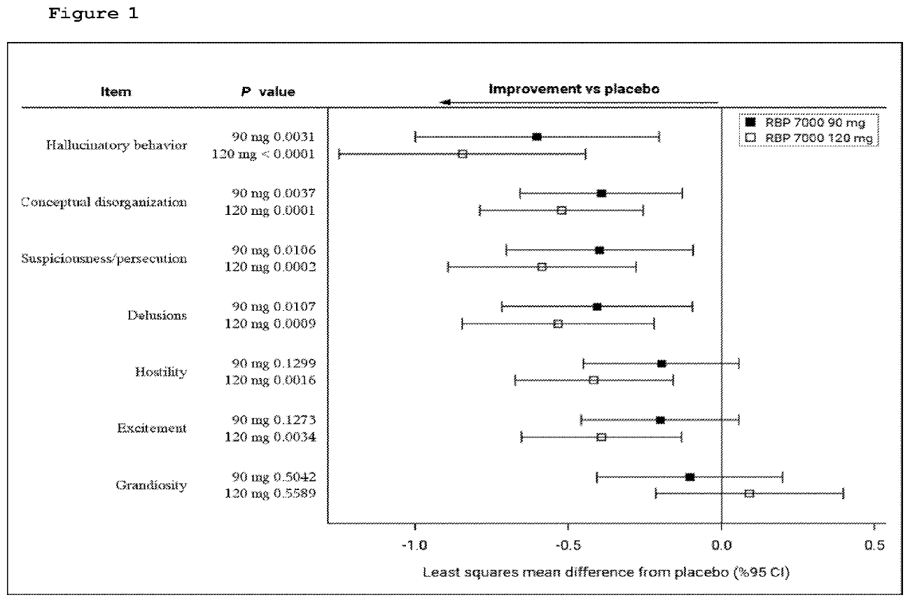

As shown in FIG. 1, Formulation C improved most symptoms on the positive subscale. In particular, treatment with either 90 mg or 120 mg of Formulation C improved four PANSS positive symptoms including: hallucinatory behavior (90 mg, P<0.01; 120 mg, P<0.0001), conceptual disorganization (90 mg, P<0.01; 120 mg, P<0.001), delusions (90 mg, P<0.05; 120 mg, P<0.001), and suspiciousness/persecution (90 mg, P<0.05; 120 mg, P<0.001). Significant improvements in hostility (P<0.01) and excitement scores were also observed with 120 mg of Formulation C when compared to placebo (P<0.01).

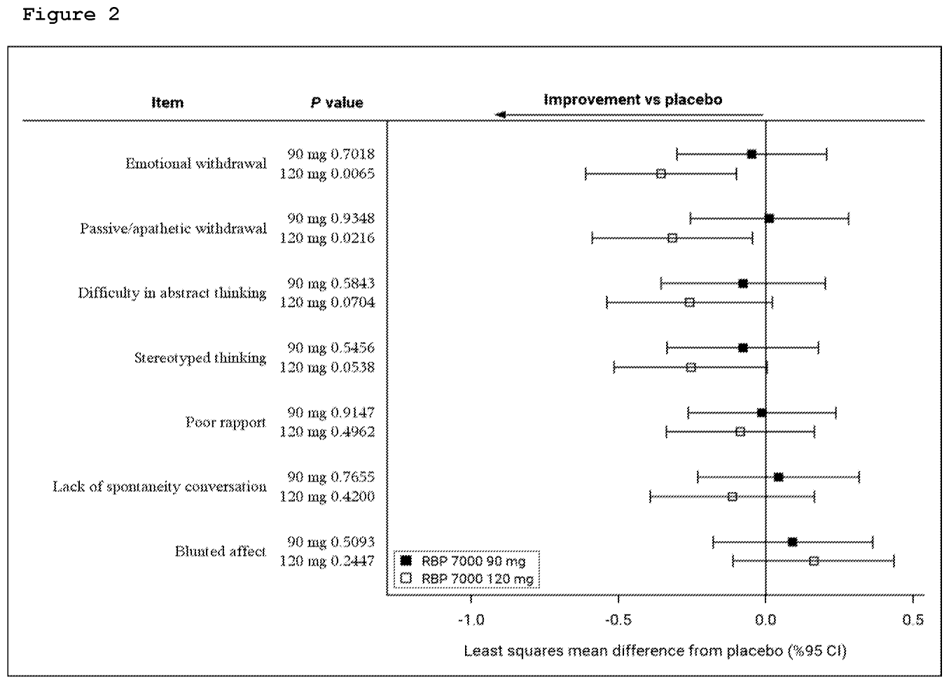

As shown in FIG. 2, Treatment with 120 mg of Formulation C improved two PANSS negative symptom scores as follows: emotional withdrawal (P<0.01) and passive/apathetic withdrawal (P<0.05). In addition, there were improvements on stereotyped thinking (P=0.054) and difficulty in abstract thinking (P=0.07) with 120 mg of Formulation C.

The 90 mg and 120 mg doses of Formulation C improved six PANSS general symptom scores when compared to placebo, as follows: depression (90 mg, P<0.001; 120 mg, P<0.0001), active social avoidance (90 mg, P<0.05; 120 mg, P<0.0001), uncooperativeness (90 mg, P<0.05; 120 mg, P<0.01), poor attention (90 mg, P<0.05; 120 mg, P<0.01), anxiety (90 mg, P<0.01; 120 mg, P<0.05), and tension (90 mg, P<0.05; 120 mg, P<0.05). Improvements in poor impulse control (P<0.001) and somatic concern (P<0.05) were also observed with 120 mg of Formulation C, while 90 mg of Formulation C also improved motor retardation (P<0.01).

Importantly, treatment with the 120 mg dose of Formulation C resulted in greater improvement on two PANSS negative symptoms (i.e., emotional withdrawal and passive/apathetic withdrawal), indicating that the 120 mg dose is useful in addressing difficult to treat negative symptoms (see FIG. 2).

While various embodiments and aspects are shown and described herein, it will be obvious to those skilled in the art that such embodiments and aspects are provided by way of example only. Numerous variations, changes, and substitutions will now occur to those skilled in the art. It should be understood that various alternatives to the embodiments described herein may be employed.

1. A method of treating a negative symptom in a schizophrenic patient in need thereof, the method comprising administering to the patient a therapeutically effective amount of a risperidone composition; wherein the risperidone composition comprises about 120 mg of risperidone base.

2. The method of claim 1, wherein the negative symptom is blunted affect, emotional withdrawal, poor rapport, passive/apathetic social withdrawal, difficulty in abstract thinking, lack of spontaneity and flow of conversation, stereotyped thinking, or a combination of two or more thereof.

3. The method of claim 1, wherein the negative symptom is emotional withdrawal, passive/apathetic social withdrawal, difficulty in abstract thinking, stereotyped thinking, or a combination of two or more thereof.

4. The method of claim 1, wherein the negative symptom is emotional withdrawal, passive/apathetic withdrawal, or a combination thereof.

5. The method of claim 1, comprising subcutaneously administering the risperidone composition to the patient.

6. The method of claim 1, comprising subcutaneously administering the risperidone composition to the patient once per month.

7. The method of claim 1, comprising subcutaneously administering the risperidone composition to the patient once every two months or once every three months.

8. The method of claim 1, wherein the risperidone composition comprises the risperidone base, a poly(lactide-co-glycolide) copolymer, and methyl-2-pyrrolidone.

9. The method of claim 8, wherein the risperidone composition comprises about 15 wt % of the risperidone base; about 38 wt % of the poly(lactide-co-glycolide) copolymer; and about 47 wt % N-methyl-2-pyrrolidone.

10. The method of claim 8, wherein the poly(lactide-co-glycolide) copolymer is a 50:50 to 90:10 poly(lactide-co-glycolide) copolymer.

11. The method of claim 8, wherein the poly(lactide-co-glycolide) copolymer is an 80:20 poly(lactide-co-glycolide) copolymer.

12. The method of claim 8, wherein the poly(lactide-co-glycolide) copolymer has a number average molecular weight from about 20,000 Daltons to about 30,000 Daltons.

13. The method of claim 8, wherein the poly(lactide-co-glycolide) copolymer comprises a carboxy terminal group.

14. The method of claim 8, wherein the risperidone composition comprises about 5 wt % to about 25 wt % risperidone base; about 25 wt % to about 50 wt % of a poly(lactide-co-glycolide) copolymer; and about 35 wt % to about 60 wt % N-methyl-2-pyrrolidone.

15. The method of claim 8, wherein the risperidone composition comprises about 10 wt % to about 20 wt % risperidone base; about 35 wt % to about 45 wt % of a poly(lactide-co-glycolide) copolymer; and about 40 wt ¾ to about 50 wt % N-methyl-2-pyrrolidone.

16. The method of claim 8, wherein the risperidone composition comprises about 10 wt % to about 30 wt % of risperidone base; about 10 wt % to about 80 wt % of a biodegradable polymer; and about 10 wt % to about 80 wt % of an organic solvent.

17. The method of claim 16, wherein the biodegradable polymer is a poly(lactide-co-glycolide) copolymer; and wherein the organic solvent is N-methyl-2-pyrrolidone.

18. The method of claim 14, wherein the poly(lactide-co-glycolide) copolymer is a 50:50 to 90:10 poly(lactide-co-glycolide) copolymer.

19. The method of claim 18, wherein the poly(lactide-co-glycolide) copolymer is an 80:20 poly(lactide-co-glycolide) copolymer.

20. The method of claim 14, wherein poly(lactide-co-glycolide) copolymer has a number average molecular weight from about 1,000 Daltons to about 50,000 Daltons.

21. The method of claim 20, wherein poly(lactide-co-glycolide) copolymer has a number average molecular weight from about 20,000 Daltons to about 30,000 Daltons.

22. The method of claim 14, wherein the poly(lactide-co-glycolide) copolymer comprises a carboxy terminal group.
